(19) United States
(12) Patent Application Publication
(10) Pub. No.: US 2026/0227399 A1
(43) Pub. Date: Aug. 6, 2026

(54) METHODS AND RELATED ASPECTS FOR THE DETECTION OF ANTIBODIES

(71) Applicant: ARIZONA BOARD OF REGENTS ON BEHALF OF ARIZONA STATE UNIVERSITY, Scottsdale, AZ (US)

(72) Inventors: Chao Wang, Chandler, AZ (US); MD Ashif Ikbal, Tempe, AZ (US); Seyedsina Mirjalili, Tempe, AZ (US)

(73) Assignee: ARIZONA BOARD OF REGENTS ON BEHALF OF ARIZONA STATE UNIVERSITY, Scottsdale, AZ (US)

(21) Appl. No.: 18/868,993

(22) PCT Filed: May 26, 2023
(86) PCT No.: PCT/US2023/067556

§ 371 Date: Nov. 25, 2024

Related U.S. Application Data
(60) Provisional application No. 63/346,741, filed on May 27, 2022.

(51) Int. Cl. G01N 33/569; G01N 21/552; G01N 33/543; G01N 33/68

(52) U.S. Cl. CPC G01N 33/56983; G01N 21/554; G01N 33/54346; G01N 33/6854; G01N 2333/165; G01N 2469/20

(57) ABSTRACT

Methods of analyzing antibodies in samples are provided. In some embodiments, the methods include contacting a sample with a plurality of plasmonic metal nanoparticles (MNPs) having a viral antigen binding moiety bound to its surface and a viral antigen that comprises two or more epitopes that bind to the viral antigen binding moiety to form a binding composition. In these embodiments, the methods include detecting an extinction signal intensity to identify antibody capabilities in the sample, including the quantity and quality of the antibody. Related systems and other methods are also provided.

### CROSS-REFERENCE TO RELATED APPLICATIONS

This application claims the benefit of and priority to U.S. Provisional Patent Application Ser. No. 63/346,741, filed May 27, 2022, the disclosure of which is incorporated herein by reference.

### STATEMENT OF GOVERNMENT SUPPORT

This invention was made with government support under 1838443 awarded by the National Science Foundation and R21 Al169098 awarded by the National Institutes of Health. The government has certain rights in the invention.

### BACKGROUND

The new coronavirus disease (COVID-19) is an unprecedented threat to human health and economics in Arizona. As of mid February 2022, ~2 million cases and >27,000 deaths have been reported in Arizona, including >600,000 cases and >4,000 deaths in two months during the Omicron surge. The omicron variant is capable of escaping from neutralizing antibodies (nAbs) after natural infection or following vaccination. NAbs are the first responders against pathogen invasions, and can interact with other immune components to assist in pathogen clearance. The quality and quantity of the nAb response dictate functional outcomes.

Sterilizing immunity against viruses can only be accomplished by high-concentration and high-affinity nAbs that can effectively block the cellular entry of viral particles. The currently approved Pfizer-BioNTech and Moderna vaccines, as well as other candidates in clinical trials, protect against SARS-CoV-2 by raising potent nAbs targeting the SARS-CoV-2's spike protein. In addition, manufactured nAbs or those extracted from human convalescent plasma are promising treatment options to reduce viral load and alleviate severity of the disease. Importantly, the quality and quantity of the antibody response dictate clinical outcomes. For example, non-neutralizing antibodies can promote pathology in some viral infections (e.g. Dengue and SARS), resulting in an antibody-dependent enhancement (ADE) effect and promoting severe inflammation. Therefore, quantification of nAbs is important to evaluating the efficacy of different vaccine candidates and identifying efficacious batches of convalescent plasma for treatment.

Additionally, previous exposure to SARS-CoV-2 or vaccination might not guarantee total immunity in all cases, as shown by the Delta and Omicron variants. Long-term vaccine efficacy is challenged by the constant mutation of SARS-CoV-2. The rise of novel variants, including potential vaccine escape Omicron variants, requires a rapid means to assess the breadth of coverage for nAbs. Therefore, frequent evaluation of the protective nAbs against emerging viral mutations is important to timely adjustment of patient treatment plans. Neutralizing assays are, therefore, important for evaluating the effectiveness of nAbs for vaccine development and immunity evaluation.

Many of the available neutralizing assays involve propagation of viruses and require a biosafety level 3 (BSL3) lab settings, which, unfortunately, are unavailable to many researchers or clinicians. Surrogate ELISA neutralizing assays have been developed to measure antibody-mediated blockage of ACE2 receptor binding to SARS-CoV-2 S protein in relaxed BSL2 labs. However, ELISA is still a lab-based process that requires multistep workflow, hours of operation, professional training, and complex readout system. Currently, there is a lack of easy-to-use, inexpensive and accurate neutralization assays. Innovations in low-cost and high-throughput nAb assay platform is very desirable to measure nAbs against different variants of concerns (VoCs).

Accordingly, there is a need for additional methods, and related aspects, for conducting neutralizing antibody assays, including those related to SARS-CoV-2.

### SUMMARY

In one aspect, the present disclosure provides a method of analyzing a sample (e.g., to measure the amount of antibodies specific to a viral infection). The method includes contacting the sample with a first plurality of plasmonic metal nanoparticles (MNPs) having a viral antigen bound to its surface and a second plurality of MNPs having a viral antigen binding moiety bound to its surface to form a binding composition. The viral antigen comprises at least one target epitope and the viral antigen binding moiety specifically binds to the target epitope. The method also includes detecting a detectable signal from the binding composition that is indicative of the presence of a neutralizing antibody in the sample, the presence of a non-2 neutralizing antibody in the sample, or the absence of the neutralizing or the non-neutralizing antibody in the sample.

In another aspect, the present disclosure provides a method of analyzing a sample. The method contacting the sample with a first plurality of plasmonic metal nanoparticles (MNPs) having a viral antigen bound to its surface and a second plurality of MNPs having a viral antigen binding moiety bound to its surface to form a binding composition, wherein the viral antigen comprises at least one target epitope and wherein the viral antigen binding moiety specifically binds to the target epitope. The method also includes detecting a first, a second, or a third MNP extinction wavelength from the binding composition, wherein the first MNP extinction wavelength is indicative of the presence of a neutralizing antibody in the sample, wherein the second MNP extinction wavelength is indicative of the presence of a non-neutralizing antibody in the sample and wherein the third MNP extinction wavelength is indicative of the absence of the neutralizing or the non-neutralizing antibody in the sample, thereby analyzing the sample.

In another aspect, the present disclosure provides a method of analyzing an antibody in a sample. The method includes contacting a first aliquot of the sample with a first plurality of plasmonic metal nanoparticles (MNPs) having a first viral antigen variant bound to its surface and a second plurality of MNPs having a viral antigen binding moiety bound to its surface to form a first binding composition, wherein the viral antigen binding moiety binds to the first viral antigen variant. The method also includes contacting a second aliquot of the sample with a third plurality of MNPs having a second viral antigen variant bound to its surface and the second plurality of MNPs having the viral antigen binding moiety bound to its surface to form a second binding composition, wherein the viral antigen binding moiety binds to the second viral antigen variant. In addition, the method also includes detecting a first MNP extinction wavelength from the first binding composition and a second MNP extinction wavelength from the second binding composition, wherein the first MNP extinction wavelength is indicative of a level of interaction of the antibody with the first viral antigen variant and wherein the second MNP extinction wavelength is indicative of a level of interaction of the antibody with the second viral antigen variant, thereby analyzing the antibody in the sample.

In another aspect, the present disclosure provides a method of analyzing an antibody in a sample. The method includes contacting the sample with a plurality of plasmonic metal nanoparticles (MNPs) having a viral antigen binding moiety bound to its surface and viral antigen compound that comprises two or more epitopes that bind to the viral antigen binding moiety to form a binding composition. The method also includes detecting an MNP extinction wavelength from the binding composition, which MNP extinction wavelength is indicative of a level of interaction of the antibody with the viral antigen compound, thereby analyzing the antibody in the sample.

In another aspect, the present disclosure provides a method of analyzing a sample to measure the amount of neutralizing antibodies that can protect the host from future viral infection, the method comprising: a solution contacting the sample for detection, further comprising the following parts: a first plurality of plasmonic metal nanoparticles (MNPs) having a viral antigen binding receptor (or motif, or moiety) bound to its surface that strongly binds to the target viral antigen to initiate viral infection in human cells, and a viral antigen with multiple binding moiety to the protein receptor, wherein the viral antigen comprises at least one target epitope that binds to the protein receptor; and detecting a detectable signal from the amount of MNPs floating in biological buffers from the binding composition that is indicative of the presence of a neutralizing antibody in the sample, or the absence of the neutralizing antibody in the sample, thereby analyzing the sample. In some embodiments, the viral antigen includes multiple binding sites to the antigen-binding receptor or moiety, further comprising: a dimer or oligomer structure that comprises two or more copies of the viral antigen, a second plurality of MNPs with multiple viral antigens bound to its surface, and/or a protein carrier with multiple viral antigens bound to its surface.

In some embodiments, the first and second pluralities of MNPs comprise a size and shape suitable for colorimetric, spectrometric, or electronic detection. In some embodiments, the first and second plurality of MNPs differ from one another. In some embodiments, for example, the first plurality of MNPs are AuNPs and the second plurality of MNPs are AgNPs. In some embodiments, the viral antigen comprises a dimer or oligomer structure that comprises two or more copies of the viral antigen. In some embodiments, the method further includes determining a quantity or a concentration of the neutralizing antibody in the sample when the detectable signal from the binding composition is indicative of the presence of the neutralizing antibody in the sample. In some embodiments, the method further includes determining a quantity or a concentration of the non-neutralizing antibody in the sample when the detectable signal from the binding composition is indicative of the presence of the non-neutralizing antibody in the sample. In some embodiments, the infectious disease antigen is a SARS-CoV-2 antigen variant and can be one of the following: a wild type variant or the Wuhan-Hu-1 strain, an alpha variant or B. 1.1.7 and descendant lineages, a beta variant or B. 1.351 and descendant lineages, a delta variant or B.1.617.2 and descendant lineages, a gamma variant, or P.1 and descendent lineages, an omicron variant, or B.1.1.529 and descendant lineages, and/or other past and emerging strains. In some embodiments, the measurement of extinction from first MNP extinction wavelength is indicative of the presence and quantity of an antibody specific to the viral antigen in the sample, either neutralizing or non-neutralizing, thereby analyzing the sample. In some embodiments, the measurement of extinction from first MNP extinction wavelength is indicative of the presence and quantity of a neutralizing antibody specific to the viral antigen in the sample, thereby analyzing the sample. In some embodiments, the second plurality of MNPs differ from the first plurality of MNPs in sizes, materials, and/or extinction wavelengths. In some embodiments, the second plurality of MNPs are functionalized with streptavidin protein and the viral antigens are biotinylated, and the amount of viral antigens on the MNPs are dependent on the sizes of the MNPs and proteins but vary from 10 and 10,000. In some embodiments, the protein carrier is streptavidin protein and the viral antigens are biotinylated, and the number of the viral antigens on the protein carrier varies from 2 to 4. In some embodiments, the sample is not processed prior to the contacting step and comprises a population of target antibodies in a liquid buffer. In some embodiments, the target antibodies are one of the following: mAbs for therapeutic purposes, a mixture of mAbs for therapeutic purposes, and/or antibodies produced from humoral response after prior vaccination or prior natural infection, or a combination of both. In some embodiments, the liquid buffer is one of the following: Phosphate Buffered Saline (PBS) buffer, a human serum, a human whole blood, and/or, another biological buffer for protein and antibody analysis.

In some embodiments, the two or more epitopes are identical to one another. In some embodiments, the two or more epitopes are different from one another. In some embodiments, the viral antigen compound comprises a dimer or oligomer structure that comprises two or more copies of the viral antigen. In some embodiments, the methods further comprise determining a quantity or a concentration of the neutralizing antibody in the sample when the first MNP extinction wavelength is detected from the binding composition or determining a quantity or a concentration of the non-neutralizing antibody in the sample when the second MNP extinction wavelength is detected from the binding composition. In some embodiments, the methods further comprise obtaining a sample from a subject prior to or concurrent with the contacting step. In some embodiments, the sample is not processed prior to the contacting step. In some embodiments, wherein the viral antigen is a SARS-CoV-2 antigen. In some embodiments, the SARS-CoV-2 antigen comprises an S1 subunit or receptor binding domain (RBD) of the spike (S) protein, or the S protein, or a fragment thereof. In some embodiments, the SARS-CoV-2 antigen comprises two or more chemically connected, repeating units of an S1 protein or RBD of the spike (S) protein, or the S protein, or a fragment thereof. In some embodiments, the viral antigen binding moiety is a SARS-CoV-2 antigen binding moiety. In some embodiments, the SARS-CoV-2 antigen binding moiety comprises an angiotensin-converting enzyme 2 (ACE2), or a fragment thereof.

In another aspect, the present disclosure provides a system that includes a receptacle receiving area configured to receive at least one receptacle that comprises a binding composition as disclosed herein, a light source capable of emitting photons having an MNP extinction wavelength corresponding to one or more of the pluralities of MNPs disclosed herein, and a photodetector capable of detecting transmitted light from one or more of the pluralities of MNPs disclosed herein.

In some embodiments, the MNPs comprise gold, silver, copper, aluminum, platinum, and palladium, or any combinations thereof. In some embodiments, the MNPs comprise a size and shape suitable for colorimetric, spectrometric, or electronic detection. In some embodiments, the viral antigen and/or viral antigen binding moiety is bound to the MNPs via a linker. In some embodiments, the viral antigen and/or viral antigen binding moiety is bound to the MNPs via biotin to streptavidin reactions.

In some embodiments, the composition is a liquid-phase composition.

In some embodiments, the composition further comprises a sample obtained from a subject's bodily fluid.

Embodiments of the present disclosure also include a method of performing a colorimetric, spectrometric, or electronic assay using any of the compositions described herein. In accordance with these embodiments, the method comprises combining the first and second pluralities of MNPs with the sample from a subject, and detecting an altered MNP extinction wavelength corresponding to the pluralities of MNPs based on the presence or absence of the target antibody.

Embodiments of the present disclosure also include a system for performing any of the colorimetric, spectrometric, or electronic assays described herein. In accordance with these embodiments, the system comprises a receptacle for combining the first and second pluralities of MNPs with the sample from a subject, a light source capable of emitting an MNP extinction wavelength corresponding to the pluralities of MNPs, and a photodetector capable of detecting transmitted light from the pluralities of MNPs.

In some embodiments, the system further comprises a means for determining a voltage and/or current readout corresponding to the transmitted light detected by the photodetector.

### BRIEF DESCRIPTION OF THE DRAWINGS

FIGS. 1A-1C schematically show aspects of a neutralizing antibody (nAb) assay development process according to one exemplary embodiment. FIG. 1A schematically shows a sensing mechanism of the nAb assay. FIG. 1B schematically shows aspects of epitope selective nAb sensing. FIG. 1C schematically depicts an exemplary high-throughput electronic readout system.

FIGS. 2A-2C schematically show aspects of a signal transduction mechanism of an nAb assay according to one exemplary embodiment. FIG. 2A schematically shows a nanoparticle preparation process. FIG. 2B schematically shows aspects of antibody detection that involve antibody induced clustering of protein coated MNPs. FIG. 2C schematically shows various exemplary detectable signal detection approaches, including a quantitative electronic readout, a colorimetric readout, a structural analysis, and a quantitative spectrometric readout.

FIGS. 3A and 3B show aspects of an antibody detection scheme according to one exemplary embodiment. FIG. 3A shows data obtained for an antibody detection assay that involved a CR3022 antibody, while FIG. 3B shows data obtained for an antibody detection assay that involved an S35 antibody.

FIGS. 4A-4E show aspects of epitope-specific neutralizing antibody sensing according to one exemplary embodiment. FIG. 4A schematically shows assay components, including AuNPs coated with ACE2, AgNPs coated with dimer RBD, and CR3022 or S35 antibodies. FIG. 4B schematically shows non-neutralizing clustering of MNPs, whereas FIG. 4C schematically shows neutralizing clustering of MNPs. FIG. 4D are images showing bare-eye views of reaction mixtures that included various concentrations of CR3022 and S35 antibodies. FIG. 4E is a plot showing normalized extinction (A.U.; Y-axis) detected at various concentrations of CR3022 (non-neutralizing) and S35 (neutralizing) antibodies (nM; X-axis).

FIG. 5 shows a viral load effect according to one exemplary embodiment. In particular, FIG. 5 is a plot (normalized extinction (A.U.; Y-axis); antibody concentration (nM; X-axis)) showing that changing the RBD concentration shifts the detection curve.

FIGS. 6A-6D show data illustrating the differentiation of RBD variants in inhibition according to one exemplary embodiment. FIG. 6A are images showing bare-eye views of reaction mixtures that included various concentrations of S35 antibodies (SAD-S35) and AgNPs coated with wild-type (WT) RBD. FIG. 6B are images showing bare-eye views of reaction mixtures that included various concentrations of S35 antibodies (SAD-S35) and AgNPs coated with P.1 (gamma) RBD. FIG. 6C is a plot showing normalized extinction (A.U.; Y-axis) detected at various concentrations of S35 antibodies (nM; X-axis) in reactions that included WT or P.1 RBD coated MNPs. FIG. 6D is a plot showing the inhibition of SARs-CoV-2 spike RBD variants: ACE2 interaction by an anti-SARs-CoV-2 neutralizing antibody (SAD-S35) (inhibition (%)-Y-axis; SAD-S35 concentration (μg/mL-X-axis).

FIG. 7 shows a portable electronic readout system schematic according to one exemplary embodiment.

FIG. 8 shows a high-throughput well-plate based readout system schematic according to one exemplary embodiment.

FIGS. 9A-9H show a neutralization assay design with an engineered viral load.

FIGS. 10A-10C show aspects of an exemplary neutralization assay.

### DETAILED DESCRIPTION

### 1. Definitions

Unless otherwise defined, all technical and scientific terms used herein have the same meaning as commonly understood by one of ordinary skill in the art. In case of conflict, the present document, including definitions, will control. Exemplary methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting. Section headings as used in this section and the entire disclosure herein are merely for organizational purposes and are not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “severe acute respiratory syndrome coronavirus-2” or “SARS-CoV-2” refers to the coronavirus that emerged in 2019 to cause a human pandemic of an acute respiratory disease, now known as coronavirus disease 2019 (COVID-19).

As used herein, the term “subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human). In some embodiments, the subject may be a human or a non-human. In one embodiment, the subject is a human. The subject or patient may be undergoing various forms of treatment.

As used herein, the term “treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease (e.g., viral infection). A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

As used herein, a “sample,” such as a biological sample, is a sample obtained from a subject. As used herein, biological samples include all clinical samples including, but not limited to, cells, tissues, and bodily fluids, such as saliva, tears, breath, and blood; derivatives and fractions of blood, such as filtrates, dried blood spots, serum, and plasma; extracted galls; biopsied or surgically removed tissue, including tissues that are, for example, unfixed, frozen, fixed in formalin and/or embedded in paraffin; milk; skin scrapes; nails, skin, hair; surface washings; urine; sputum; bile; bronchoalveolar fluid; pleural fluid, peritoneal fluid; cerebrospinal fluid; prostate fluid; pus; or bone marrow. Samples include both those collected from living patients and those collected postmortem. In a particular example, a sample includes blood obtained from a subject, such as whole blood or serum. In another example, a sample includes cells collected using an oral rinse. Methods for diagnosing, predicting, assessing, and treating LD in a subject include detecting the presence or absence of antibodies to one or more biomarkers described herein, in a subject's sample. The sample may be isolated from the subject and then directly utilized in a method for determining the presence or absence of antibodies, or alternatively, the sample may be isolated and then stored (e.g., frozen) for a period of time before being subjected to analysis.

As used herein, the term “antibody” refers to an immunoglobulin or an antigen-binding domain thereof. The term includes but is not limited to polyclonal, monoclonal, monospecific, polyspecific, non specific, humanized, human, canonized, canine, felinized, feline, single chain, chimeric, synthetic, recombinant, hybrid, mutated, grafted, and in vitro generated antibodies. The antibody can include a constant region, or a portion thereof, such as the kappa, lambda, alpha, gamma, delta, epsilon and mu constant region genes. For example, heavy chain constant regions of the various isotypes can be used, including: IgG1, IgG2, IgG3, IgG4, IgM, IgA1, IgA2, IgD, and IgE. By way of example, the light chain constant region can be kappa or lambda. The term “monoclonal antibody” refers to an antibody that displays a single binding specificity and affinity for a particular target, e.g., epitope.

As used herein, the term “neutralizing antibody” or “nAb” refers to an antibody that protects a cell from a pathogen, such as a virus or bacterium, by eliminating the biological effect the pathogen would otherwise have on the cell without a need for immune cell intervention. Neutralization renders the pathogen no longer infectious or pathogenic. Neutralizing antibodies are part of the humoral response of the adaptive immune system against intracellular bacteria, viruses, and microbial toxins. By binding specifically to surface antigens on a given pathogen, neutralizing antibodies prevent the pathogen from interacting with host cells that the pathogen might otherwise infect.

As used herein, the term “non-neutralizing antibody” or “binding antibody” refers to an antibody that bind specifically to a pathogen, but does not directly interfere with the pathogen's infectivity. Non-neutralizing antibody binding signals immune cells that the pathogen has been targeted for destruction by the immune cells.

As used herein, the term “binding”, typically refers to a non-covalent association between or among two or more entities.

As used herein, “specifically bind,” in the context of pathogen detection, refers to a state in which substantially only target chemical structures (e.g., target SARS-CoV-2 proteins) are sufficiently associated with a corresponding or cognate binding agent (e.g., an antibody, or antigen binding portion thereof), to the exclusion of non-target chemical structures, such that the association between the target chemical structures and the binding agent can be detected.

As used herein, “detect,” “detecting,” or “detection” refers to an act of determining the existence or presence of one or more analytes in a given sample.

As used herein, “epitope” refers to the part of an antigen (e.g., a SARS-CoV-2 protein) to which an antibody and/or an antigen binding portion binds.

As used herein, “conjugate” refers to a reversible or irreversible connection between two or more substances or components. In some embodiments, for example, gold nanoparticles (AuNPs) and/or other plasmonic metal nanoparticles (MNPs) are connected to antibodies and/or to antigen binding portions thereof. In some embodiments, AuNPs and/or other plasmonic metal nanoparticles (MNPs) are conjugated with antibodies and/or to antigen binding portions thereof via one or more linker compounds.

### 2. Exemplary Neutralizing Antibody Assays and Related Aspects

In the recent past, many new viral pathogens emerged as major public health threats, including Ebola virus, severe acute respiratory syndrome coronavirus (SARS-CoV), and most recently severe acute respiratory syndrome coronavirus 2 (SARS-CoV-2). Measurement of sterilizing immunity against viruses involves quantitative and specific analysis of neutralizing antibodies (nAbs), however, such assays conventionally require stringent biosafety level lab settings and are not available to many researchers or clinicians. In addition, clinical laboratory-based antibody tests measure the total Ab level responding to SARS-CoV-2 antigens, without functionally evaluating pathogen-bound Abs and therefore cannot predict neutralizing activity. Lastly, the evolution of different virus variants has shown significant immunity-escape potential. It is important to establish quantitative and inexpensive evaluation systems of the effectiveness of nAbs against emerging viral pathogens for populations susceptible to infections and individuals with compromised immunity. Accordingly, the present disclosure provides metal nanoparticle-based rapid diagnostic platforms that are sensitive, low-cost, and epitope-specific for the detection of nAbs without requiring a high biosafety level, and are, for example, validated by detecting nAbs against the spike proteins of different SARS-CoV-2 strains.

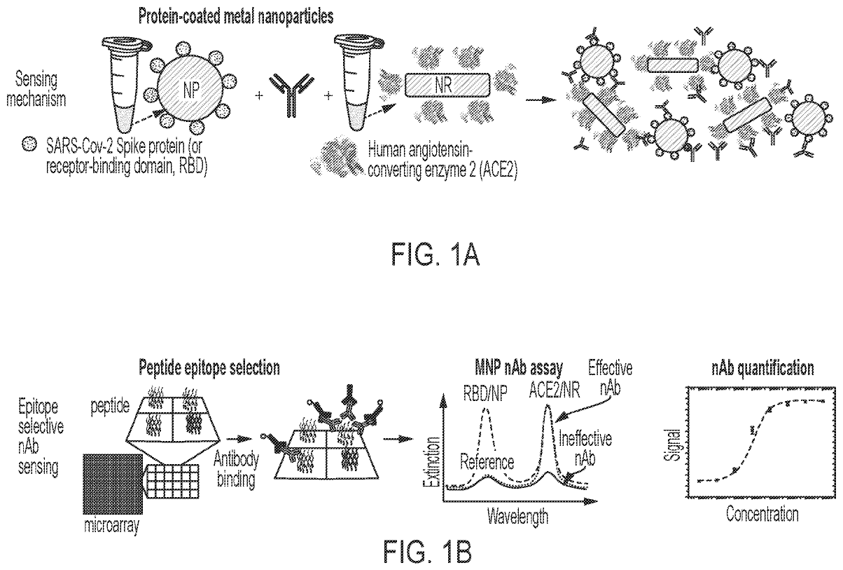

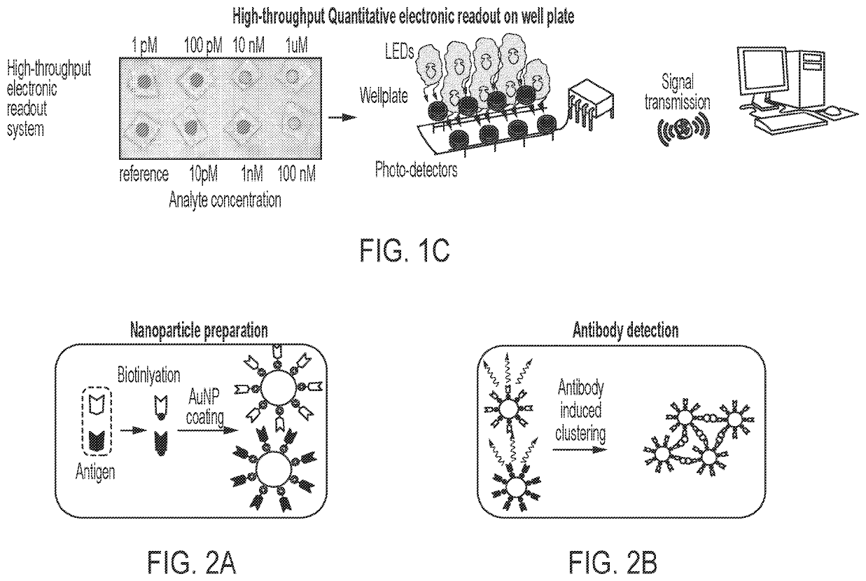

As an overview, FIGS. 1A-1C schematically show aspects of a neutralizing antibody (nAb) assay development process according to one exemplary embodiment. More specifically, FIG. 1A schematically shows a sensing mechanism of the nAb assay that involves the formation of aggregations of target antibodies with SARS-CoV-2 protein coated nanoparticles and ACE2 coated nanoparticles. FIG. 1B schematically shows aspects of epitope selective nAb sensing, which involves peptide epitope selection, MNP nAb assay development, and nAb quantification. In addition, FIG. 1C schematically depicts an exemplary high-throughput electronic readout system that uses incident LED light, microwells, and photodetectors in addition to a signal transmission mechanism.

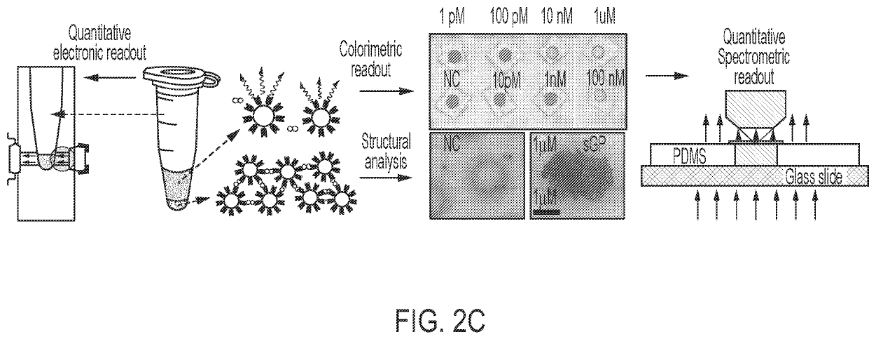

In addition, FIGS. 2A-2C schematically show aspects of a signal transduction mechanism of an nAb assay according to one exemplary embodiment. FIG. 2A schematically shows a nanoparticle preparation process that involves coating MNPs with selected antigens. FIG. 2B schematically shows aspects of antibody detection that involve antibody induced clustering of antigen/protein coated MNPs, which is discussed additionally below. Further, FIG. 2C schematically shows various exemplary detectable signal detection approaches, including a quantitative electronic readout, a colorimetric readout, a structural analysis, and a quantitative spectrometric readout, which are described further herein.

As a further illustration, an overall scheme of detecting virus-specific antibodies is shown in the aforementioned FIGS. 2A and 2B. Briefly, the Gold nanoparticles (AuNPs) are conjugated with biotinylated virus antigens, such as the full spike (S) protein or nucleocapsid (N) protein of SARS-CoV-2, or the S1 of the S protein, or the receptor-binding domain (RBD) of the S protein, or a peptide sequence on the S or N protein, or a dimer form of the above-mentioned proteins, etc. The presence of antibodies in the blood or serum will recognize the antigens and trigger the binding reaction. This causes subsequent binding of AuNP monomers together into larger aggregates that eventually precipitate. The precipitation of AuNPs will result in a change of solution color.

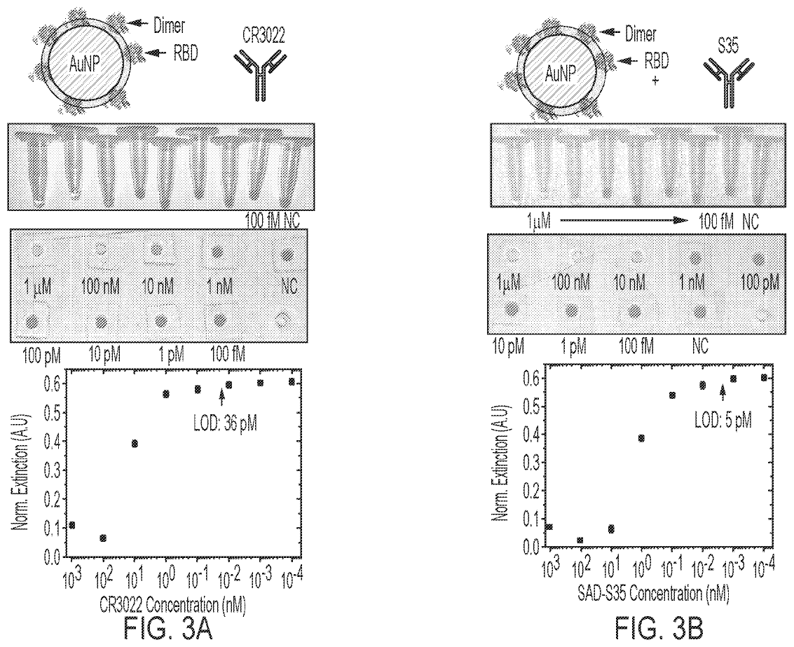

To characterize this scheme, we have tested two different high-affinity antibodies, CR3022 and S35. CR3022 is an antibody specific to the RBD protein of both SARS-COV and SARS-CoV-2. It neutralizes SARS-CoV, but does not neutralize SARS-CoV-2. S35 is a neutralizing antibody to SARS-CoV-2. In both cases (FIGS. 3A and 3B), the antibodies will trigger a significant precipitation of AuNPs, rendering more transparent solution at higher antibody concentration. The detection limit of the two antibodies are comparable, 36 pM for CR3022 and 5 pM for S35.

In this test, the end result is fewer AuNPs at high antibody concentration. This assay is effective as a serologic test with lower cost, high accuracy and faster reaction time than many existing assays, such as ELISA or lateral flow assays. However, this above test, although effective in quantifying the total antibodies against a virus, does not correlate with the amount of nAbs that can effectively block the virus cell entry. For this purpose, an epitope-specific nAb assay is used that can better evaluate the neutralizing performance in some embodiments.

In some embodiments, the epitope-specific nAb assay is implemented by introducing another set of nanoparticles that are decorated with the specific binding proteins, or peptide sequences, that are important to virus replication. In this case, we use the human angiotensin-converting enzyme 2 (ACE2) to coat AuNPs to mimic the human cells. The RBD-coated silver nanoparticles (AgNPs) are used to mimic the viruses. The ACE2 receptor bind strongly to the RBD proteins, thus triggering a clustering process of both AuNPs and AgNPs, creating a transparent solution. Strong neutralizing antibodies that bind tightly on the RBD domain prevent the RBD-ACE2 reaction, thus protecting the “human cell”-like AuNPs. This will thus free the AuNPs into the solution and produce a red color. However, for non-neutralizing antibodies that do not have strong binding affinity to the RBD (or other antigens) or that bind to the RBD strongly but do not affect the RBD-ACE2 binding, the AuNPs will not be freed, and the solution will still have a light color. Or, if the amount of nAbs is not sufficient to out-compete the RBD proteins, the solution color will remain transparent.

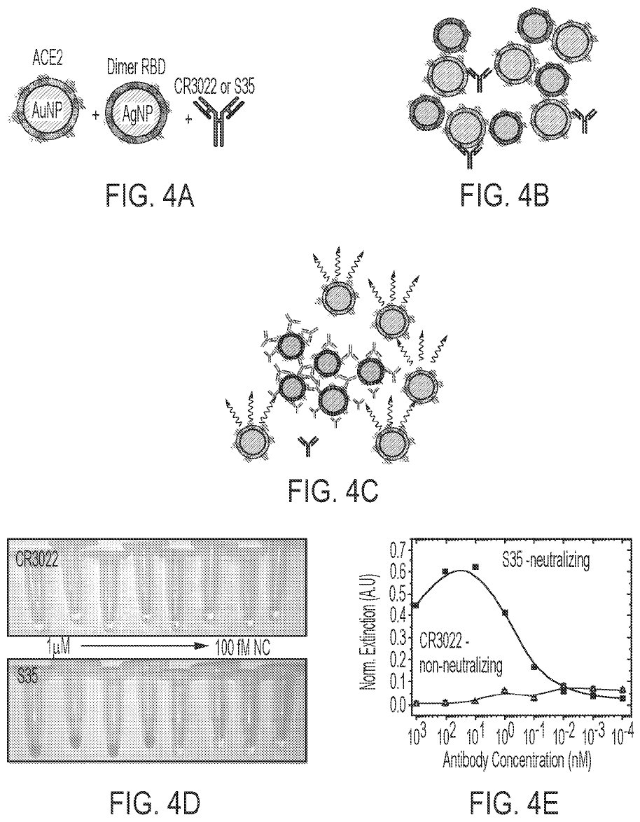

In one case (FIGS. 4A-4C), the S protein, or the N protein, or the S1 protein, or the RBD protein, or a dimer or oligomer form of such proteins, are functionalized on a set of metal nanoparticles, such as silver nanoparticles (AgNPs), to create multivalent, antigen-coated, virus-like particles. When the antibody is not capable of neutralizing the antigen-coated AgNPs, such AgNPs will react with ACE2-coated AuNPs (FIG. 4B). When the antibody is capable of neutralizing the antigen-coated AgNPs, the ACE2-coated AuNPs will be freed into the solution to produce reddish color (FIG. 4B).

In another case, the S protein, or the N protein, or the S1 protein, or the RBD protein, are connected into a dimer or oligomer form with more than one binding site. Such proteins are not conjugated with AgNPs but free in solution. Here, each of the dimer or oligomer form of such proteins is capable of binding to more than one AuNPs decorated with ACE2. When the antibody is not capable of neutralizing the dimer or oligomer antigens, such antigens will react with ACE2-coated AuNPs and cause the AuNPs to precipitate. When the antibody is capable of neutralizing the antigens, the ACE2-coated AuNPs will be freed into the solution to produce reddish color. This is evident in the comparison of CR3022 and S35, detected using such a scheme (FIGS. 4D and 4E). In the case of CR3022, the solution color is transparent for all concentrations, indicating the antibody is not neutralizing no matter how high concentration we use. However, in the case of S35, the solution color becomes reddish at higher concentration, indicating high enough concentration of S35 is able to neutralize the RBD-coated virus-like particles. Different from the assays referenced in connection with FIGS. 3A and 3B above, in this test, the end result is more AuNPs at higher nAb. This assay is effective as a nAb test to evaluate the epitopes of the nAbs that will target.

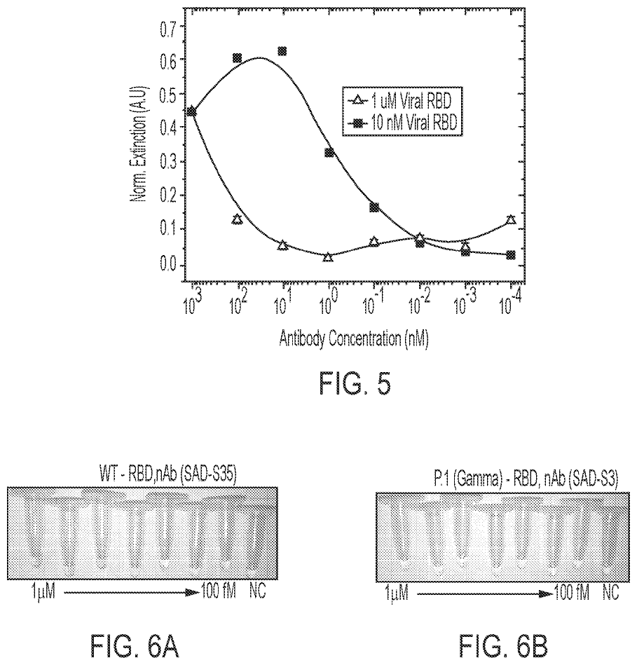

To further evaluate, we used two different concentrations of dimer RBD and tested the nAb S35 at different concentrations (FIG. 5). Clearly, for the two cases, the amount of nAbs needed for neutralizing the antibodies are dependent on the virus-like particle (i.e., dimerized RBD) concentration. This shows the feasibility of the assay in optimizing the nAb dose to neutralize viruses.

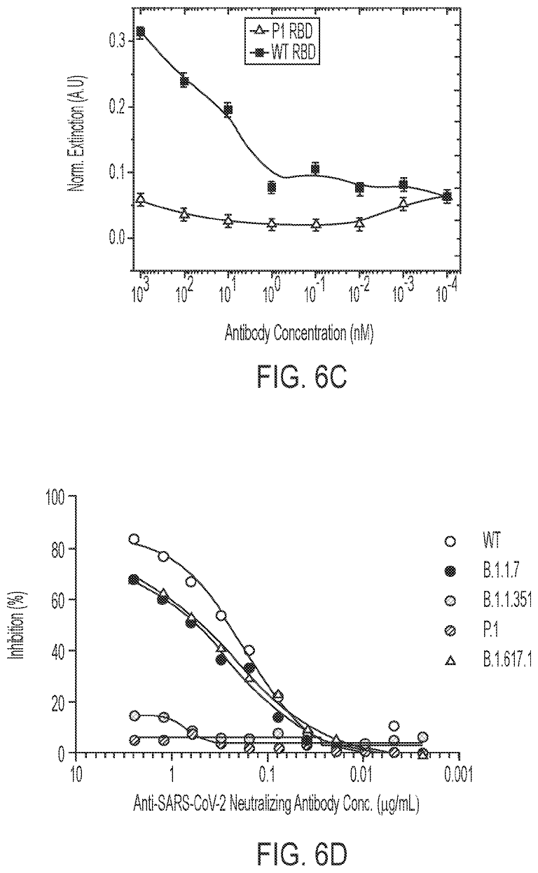

To evaluate the nAb against different variants, we chose two RBDs, the wild type and P1 variant (FIGS. 6A-6D). We used the neutralizing assay scheme as shown in FIGS. 4A-4C, where AgNPs are used as carriers to create multivalent, antigen-coated, virus-like particles. The S35 nAb is able of neutralizing the wild type but not the P1 variant according to a test done by a vendor. In our test, we observed that by increasing the S35 concentration against the wildtype RBD, AuNPs are freed, indicating neutralization. However, in the case of P1 RBD, there was no observation of significant color change at all S35 concentrations.

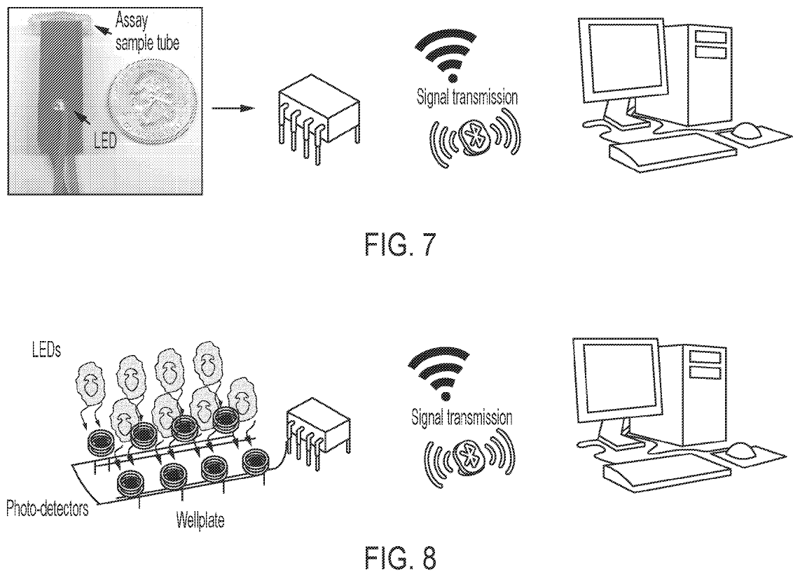

The present disclosure also provides various system for performing the assays described herein. For example, FIG. 7 schematically depicts aspects of a portable electronic system according to one embodiment. As shown, a testing tube is inserted in a holder (e.g., a 3D printed holder), where an LED and a photodetector are mounted. The light emitted from the LED transmits through the solution with free MNPs (e.g., AuNPs) and is collected by the photodetector to produce electronic signals. The signals are processed by a circuit board and transmitted to a directly connected computer, to a remote cloud-based device, and/or the like.

As an additional example, FIG. 8 schematically depicts aspects of a high-throughput well-plate reader with electronic system according to one embodiment. In this format, aliquots of top-level solutions in testing tubes are collected in a well plate. The wells are sandwiched between LEDs and photodetectors. The light emitted from the LED transmits through the solution with free MNPs (e.g. AuNPs) in a given well, and is collected by the photodetector to produce electronic signals. The signals are processed by a circuit board and transmitted to a directly connected computer, to a remote cloud-based device, and/or the like.

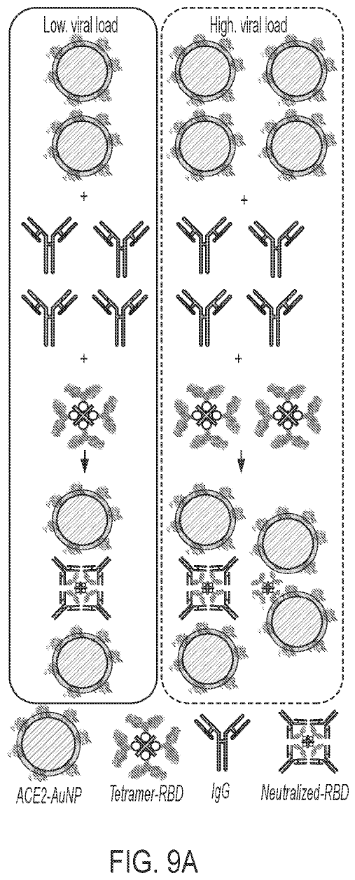

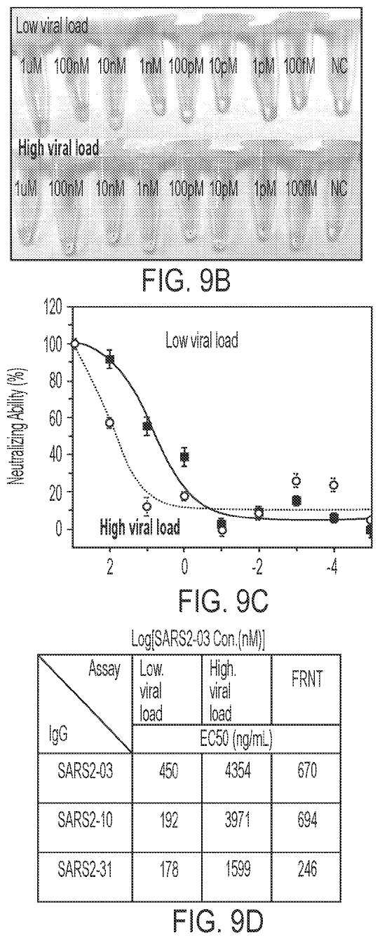

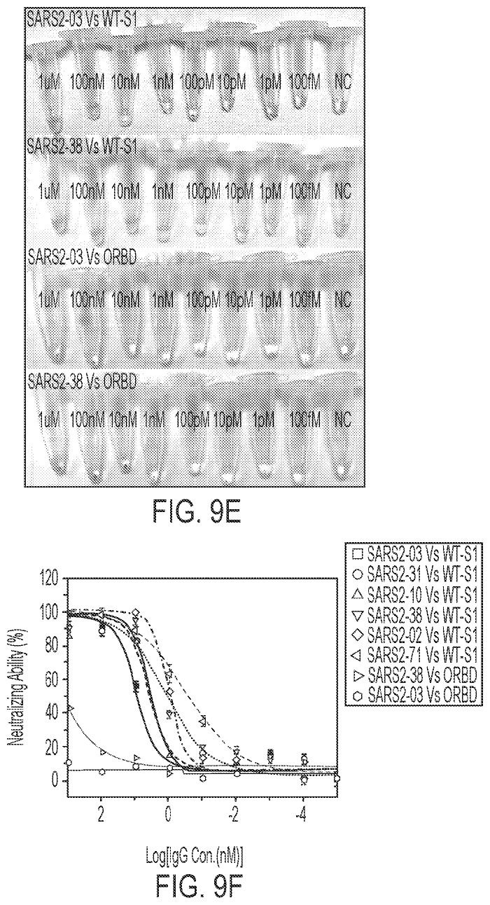

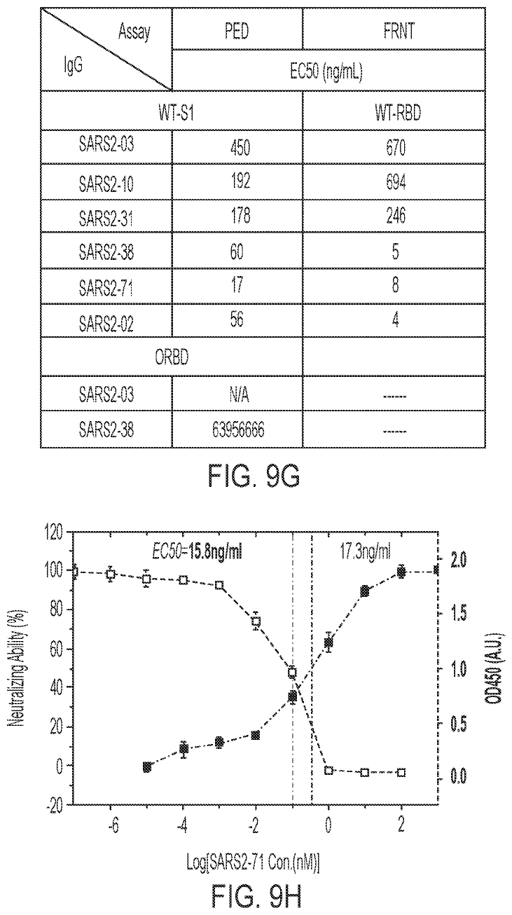

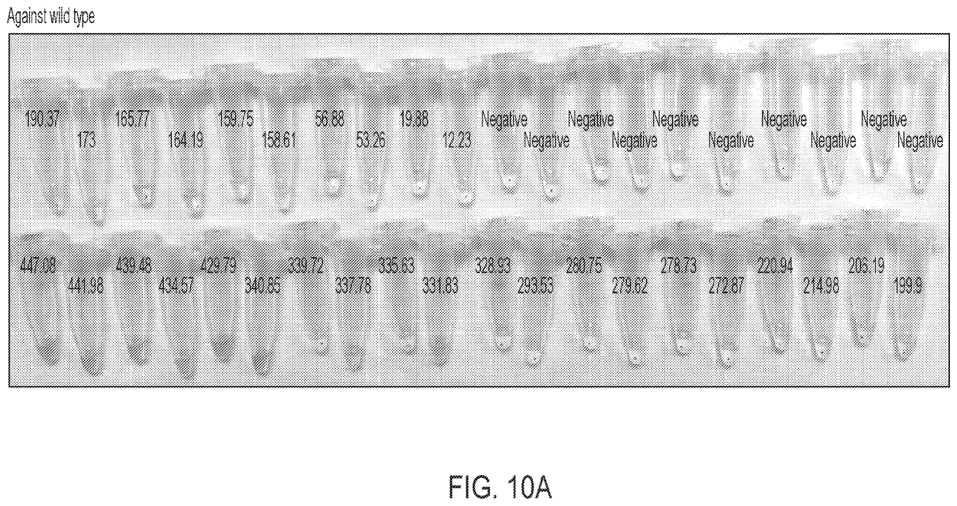

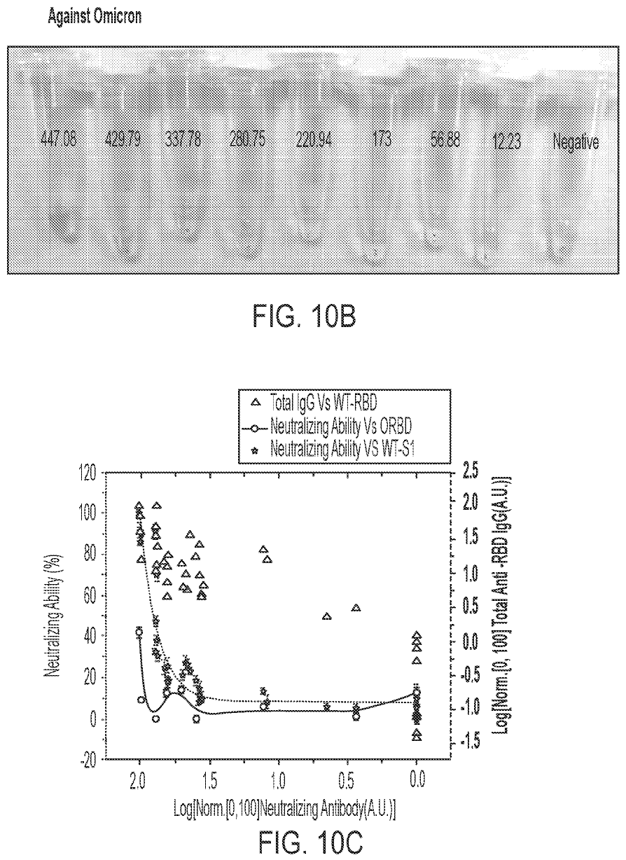

To further illustrate, FIGS. 9A-9H show aspects of a neutralization assay design with an engineered viral load. As shown, FIG. 9A is a schematic representation of the viral load/probe concentration effect on the neutralization assay. Left: the low viral load scheme; right: the high viral load scheme. In both schemes, the AuNPs are the same (e.g. 80 nm size) and functionalized with the same proteins (e.g. Omicron RBD (ORBD) or wild type S1 (WT-S1) protein, etc.). However, in the low viral load scheme, the amount of functionalized AuNPs is smaller than that in the high viral load. Here the amount of ACE2 protein functionalized AuNPs remaining floating (and therefore give the red color) is corresponding to the neutralizing capability of the tested antibody, which depends on the mAb concentration, binding to the viral protein, and competition in bonding site to the viral protein (ORBD, WT-S1, etc.) with the ACE2 protein. FIG. 9B is an optical picture of the neutralization assay for SARS2-03 monoclonal antibody (mAb) concentrations against WT-S1 in low and high viral load/probe conditions, highlight the design difference in the two sensing modes. The IgG molar concentration are marked on each tube, where NC=Negative Control. Visually, the color of high-concentration SARS-03 tubes become more dark for the low viral-load sensing mode than the high-viral load sensing mode. This is understood because more mAbs are needed to neutralize the viral WT-S1 proteins and protect the AuNPs functionalized with ACE2 receptor. FIG. 9C is a normalized electronic readout, or neutralizing ability, plotted in logarithm against SARS2-03 concentrations in low (darker trace) and high (lighter trace) viral load/probe assay conditions. The data in FIG. 10B are collected by portable electronic detector (PED). FIG. 9D shows a comparison of the EC50 value for three different SARS-COV-2 mAbs, i.e. SARS2-03, SARS2-10, SARS2-31 against wild type variant, here tested with the WT-S1 protein, in low and high viral load/probe conditions and comparison with the reference data (focus reduction neutralization test (FRNT) assay). Here, the EC50 values are calculated taking into account the concentration difference of WT-S1 proteins. The obtained EC50 values are on the same order of the FRNT assays, indicating the feasibility of the neutralizing antibody assay for quantification of neutralizing antibodies. FIG. 9E is a representative optical picture of the neutralization assay for two mAbs (SARS2-03, and SARS2-38) against the wild-type and Omicron virus (tested by WT-S1, and ORBD proteins). The assay works at low viral load/probe assay condition. IgG concentration and reaction groups indicated on each corresponding tube, NC=Negative Control). FIG. 9F is a normalized electronic readout (Neutralizing ability) of selected mAbs (SARS2-03,38,71,02,10,31), with the data for SARS2-03 and SARS2-38 corresponding to FIG. 9E, plotted in logarithm against the mAb concentrations against WT-S1,ORBD. The assay works at low viral load/probe assay condition (highlighting the capability of the neutralizing assay for different virus variants). FIG. 9G shows an EC50 value extracted from FIG. 9F for mAbs against wild type and omicron variant, compared with the reference data from FRNT assay. Here, the EC50 values are calculated taking into account the concentration difference of WT-S1 proteins. The obtained EC50 values are on the same order of the FRNT assays despite the neutralizing capability of the mAbs are orders of magnitude different. This indicates the success of the neutralizing antibody assay with PED readout can be useful to quantify the neutralizing antibodies against different virus variants. FIG. 9H shows the EC50 value from the neutralizing assay by PED readout (black, in low viral load sensing scheme) compared with ELISA neutralizing assay (lighter grey), using mAb SARS2-71 against WT variant as an example. (EC50 values adjusted to the molar ratio of RBD concentration for comparison). Here a low signal from ELISA indicates a high neutralizing capability, whereas in our neutralizing assay a high signal indicates a high neutralizing capability. In general, the trends of ELISA correlate well with the nanoparticle neutralizing assay, and the derived EC50 values are very close, i.e. 15.8 ng/ml and 17.3 ng/ml, respectively. This indicates the neutralizing assay we develop here has a comparable performance and accuracy compared to ELISA. However, ELISA requires much longer time and higher cost, as well as more personnel training.

As an additional illustration, FIGS. 10A-10C show aspects of a neutralization assay. As shown, FIGS. 10A and 10B show optical pictures of the neutralization assay for 40 serum samples (10 Negative+30 Positive) against WT-S1 and ORBD in minimum viral load/probe conditions. The labels indicate the reported neutralizing antibody values (arbitrary units) by neutralizing ELISA assay. FIG. 10C is a plot showing a comparison of neutralizing antibody assay with a total IgG antibody assay. Here, the normalized electronic readout in neutralizing ability (0 to 100%, left Y axis) against WT-S1 (fitting and star traces) and ORBD (trace with spheres). from our assay is plotted against normalized ELISA-reported neutralizing antibody concentration (A.U. x axis, in log). In addition, the total Anti-RBD IgG count (A.U.) obtained from ELISA is also plotted on the right y axis and in the triangles. Clearly, there is a consistent trend between the neutralizing antibody capability detected from our assay (stars) when it is fitted in sigmoidal function against the ELISA measured neutralizing antibody concentration. However, the measured total IgG, which is performed by many antibody assays, have a large deviation, attributed to the discrepancy between the amount of RBD-binding antibodies and the neutralizing capability. On the other hand, the neutralizing capability of the serum samples significantly decreased when compared to Omicron variant. This also indicates the feasibility of our assay to monitor the level of immunity protection as the virus variants change.

Various customized portable electronic readout systems are optionally utilized. In some embodiments, a portable readout system has a format of an enclosed container, with tube holders (e.g., 3D printed tube holders) on a top lid that accommodate testing tubes (e.g, 1, 2, 3, 4, 5, 6, 7, 8, 9, 10, 15, 20, 30, 40, 50, or more tubes). This design includes an array of LEDs and photodetectors for each testing tubes. The pairs of LEDs and photodetectors are powered by a circuit board. The circuit board can be powered, for example, by plugging into the wall or being electrically connected to a battery. In some embodiments, the circuit board has wifi and Bluetooth modules to send the signals to a wirelessly connected computer.

In some embodiments, electronic signal transduction is accomplished using commercially available microprocessors. In one exemplary embodiment, a commercially available Arduino Uno microprocessor is used as the circuit board. The overall system can include multiple modules, including a signal transduction module, a signal processing module, and a signal output module. The signal transduction module typically comprises LEDs and photodetectors to interface with testing tubes. The signal processing module typically comprises the circuit board, battery, wire connections, etc. In some embodiments, the signal output module has an LCD display, a push button to initiate a given test, multiplexers, and Bluetooth® modules for signal transmission.

In some embodiments, electronic signal transduction is accomplished using customized circuit boards. In some of these cases, a PCB board is customized with different circuit elements for signal processing. In these embodiments, the overall system typically has the same signal transduction module, signal processing module, and the signal output module as described above. Customized circuit boards can have the advantage of further lowering cost, decreasing the system footprint, reducing noise, and allowing more modes of signals processing and data transmission.

In some embodiments, a user interface is used to allow easy access to data. In some embodiments, for example, multiple windows and menus are included to guide users through the test process. In some embodiments, data received from a circuit board is automatically transmitted to a database, and the data can be retrieved and displayed in a real time. In addition, the data output can be calibrated, and used to interpret the virus load, the effectiveness of nAbs, etc. It also allows multiplexed detection at different light wavelengths.

The innovative nanosensor platforms and related assays of the present disclosure includes various features distinguishing them from previous technologies. To exemplify, the metal nanoparticle (MNP assays) are quantitative and accurate, with a dynamic range of 3 to 6 logs and a detection limit in the picomolar to femtomolar range, depending on the choice of the proteins and antibodies. Further, this assay can be implemented in a rapid detection format without any washing steps, thus significantly simplifying its operation, reducing assay time to a few minutes, and making it feasible for mass-testing. Importantly, the assay is capable of detecting virus variants by targeted binding to the epitopes on the spike protein that are sensitive to mutations. Additionally, the readout can be performed in a portable system or a high-throughput well plate format with added electronic components, making the system automated in both detection and data analysis. This rapid and quantitative nAb assay can help to timely determine the potential best uses of convalescent plasma and antibody treatment with future emerging novel viruses. Its low cost, simple operation, and automation capability are also very useful in longitudinal studies of the immune response related to, for example, COVID-19 infection, vaccination, and potential viral escape due to mutations. Among other attributes, the nAb sensing can also be used for large-population sero-surveillance in determining the level of population-based immunity (herd immunity) against any virus strains.

The low cost, simple operation, and automation capability make the assays and other aspects disclosed herein very accessible in both labs and point-of-care use. The nAb sensing is very useful in longitudinal studies of the immune response related to COVID-19, and can be used for determining the level of vaccination-elicited immunity against any emerging virus strains, for example, for seniors in long-term care facilities or immune-compromised individuals. The systems disclosed herein can make a clinical impact by rapidly identifying individuals with inadequate immune responses necessitating special vaccination or medication schedules. The modular nature of the assays enables the RBD or peptide epitopes to be changed as novel VOC (e.g. Omicron variant) or other viruses emerge. The quantitative determination of nAbs concentrations needed to neutralize virus variants at different viral loads can also be used to assist the development of individualized treatment plan for those who are infected. In addition, the feasibility of standardizing nAb measurements benefit the administration of convalescent plasma and antibody therapy to patients in case of emergence of novel viral diseases.

Embodiments of the present disclosure provide plasmonic metal nanoparticle (MNP) based colorimetric, spectrometric, or electronic assays to identify and quantify COVID19-related antibodies using optical and electronic readouts. Analytes (e.g., immunoglobulins including neutralizing antibodies) modulate the extent of MNP clustering and precipitation, and accordingly, changes the suspension color and intensity, which can be quantified to determine the concentration, binding affinity, and even binding epitope of the analyte. The present disclosure has the capability to substantially promote the availability of serology tests and assist the diagnosis, vaccination, and treatment of COVID-19 disease.

In accordance with this, embodiments of the present disclosure provide a portable colorimetric, spectrometric, or electronic sensor design for rapid detection of COVID-19 antibodies, including different types of immunoglobulins (IgG, IgM, IgA, etc.), synthetic antibodies such as but not limited to nanobodies, and virus-specific neutralizing antibodies (nAbs). In some embodiments, different assay variants can be used, including MNP in suspension and dried states (bare-eye readout), spectroscopic quantification, and optical and structural analysis. In some embodiments, the MNP shape and size, analyte and MNP concentration, and binding affinity affect the limit of detection, dynamic range, and assay time will be incorporated into the assays of the present disclosure. Additionally, immunoglobulin and virus-specific ligands can be conjugated, such as anti-IgG (or anti-IgM), the receptor-binding domain (RBD) from SARS-CoV-2 spike protein, and peptide ligands derived from nAb epitope characterization studies, on MNPs of different geometries and materials that display distinct colors. Such heterogeneous MNPs can be used to establish a sandwich-type assay capable of detecting multiple types of antibodies by bare eyes. Additionally, a competitive assay can be developed that includes heterogeneous MNPs surface-conjugated with RBD and human angiotensin-converting enzyme 2 (ACE2), a cell receptor responsible for SARS-CoV-2 infection. Effective nAbs compete with ACE2-bound MNPs in RBD binding to prevent the clustering of such MNPs, while ineffective nAbs cause MNP precipitation and change in the assay color. As would be recognized by one of ordinary skill in the art based on the present disclosure, the compositions, assays, and systems described herein can be used with any SARS-CoV-2 antigen recognized by antibodies in a sample. In some aspects, the present disclosure includes references to the analysis and detection of Ebola virus proteins (e.g., sGP). These aspects are included, for example, to further illustrate certain general principles of the assay formats disclosed herein that are optionally adapted for use in the analysis and detection of COVID-19 antibodies.

### 3. Detection Assays Using Metal Nanoparticles

Embodiments of the present disclosure also include a new plasmonic metal nanoparticle (MNP) based colorimetric, spectrometric, or electronic assay platform that will support a variety of sensing schemes, including multiplexed detection of SARS-CoV-2 immunoglobulins and validating the efficacy of potent nAbs. Using different assay variants, such as MNP in suspension (e.g., in microcentrifuge tubes or customized PDMS well plate) and dried states (e.g., on glass or gold surface), structural analysis and optical detection are combined with intuitive physical pictures and a theoretical mathematical model to comprehensively understand the mechanisms of MNP-based multivalent analyte-binding in antibody sensing. Such studies will build a foundation to further incorporate heterogeneous MNPs displaying distinct colors from blue to red to improve specificity, achieve multiplexed detection, and expand assay functionalities. In addition, a portable and inexpensive detecting instrument will be developed that provides more precise quantification than bare-eye readout, feasible for clinical settings and field deployment.

Embodiments of the present disclosure enable a comprehensive understanding of antibody-sensing mechanisms using MNP-based colorimetric, spectrometric, or electronic assays, experimentally determined assay performance, and a complete suite of antibody sensing solution without requiring lab instruments or personnel training. The assay platform provided herein will facilitate inexpensive, fast, and accurate antibody detection/quantification that can evaluate protective immune responses in individuals who have recovered from COVID-19 infection or who are at high risk of new infection. These assays can be used to evaluate the efficacy, strength, and duration of vaccines that are under development or in clinical trials. Additionally, understanding MNP-based sensing mechanisms will establish functional assay formats and demonstrated sensor performance, which will serve to accelerate the design of other POC tests for diagnosis and treatment of COVID-19 disease.

SARS-CoV-2 virions are spherical nanoparticles of about 100 nm with a membrane envelope that is studded with homotrimers of the spike (S) glycoprotein (FIG. 2). S proteins are post-translationally cleaved in the secretory pathway to yield N- and C-terminal S1 and S2 subunits, respectively. S1 is organized into an N-terminal domain (NTD), a central receptor-binding domain (RBD), and a C-terminal domain (CTD). The S1 RBD engages the viral receptor, human angiotensin-converting enzyme 2 (ACE2), at the host cell surface, followed by S protein cleavage by the transmembrane protease serine protease-2 (TMPRSS2) at the cell surface, as well as in endosomes. This cleavage activates S2 conformational rearrangements that catalyze the fusion of viral and cellular membranes and escape of the viral genome into the cytoplasm, which initiates disease-causing cycles of viral replication. Following infection, most individuals will develop an immune response to the virus, including the production of neutralizing antibodies that can prevent future infection by blocking the binding activity of the S glycoprotein. Therefore, the S glycoprotein is the major antigenic target on the virus for protective antibodies, and is thus of high significance for diagnostics as well as the development of vaccines and therapeutic antibodies. Current COVID-19 diagnosis is mainly based on epidemiological history, clinical manifestations and biomolecular marker detection. At present, real-time quantitative polymerase chain reaction (RT-qPCR) that identifies the viral RNA SARS-CoV-2 is most widely used. Yet, the PCR assay does not provide information regarding the immune response.

Antibody-based detection, such as by enzyme-linked immunosorbent assay (ELISA), has shown the feasibility of detecting IgM and IgG antibodies in serum, which indicate the short-term and long-term immune response to pathogens. Studies with SARS-CoV-2 and other human CoVs demonstrate a marked transition from seronegative to seropositive for both Ig and IgM occurs about 9 days after the onset of symptoms. These serological tests, although not ideal for early detection of viral infection, serve to identify recent and past infections and to conduct population-level surveillance, which is critical to understanding the transmission, pathogenesis, mortality rate, and epidemiology of SARS-CoV-2 viruses. Many of the commercially approved tests are lateral flow assays (LFA), which involves running the fluid containing antibodies (patient blood) over a solid substrate containing SARS-CoV-2 antigens. If the antibodies are present in the blood, they will bind the viral protein and cause a color change indicating a positive test. The LFA test, based on simple positive or negative detection of antibodies, is useful for large scale surveillance, but does not provide any information regarding the amount, type, or function of the antibodies. A better test for accurately detecting antibodies against SARS-CoV-2 is the enzyme-linked immunosorbent assay (ELISA), a common laboratory test that can measure not only the presence but also the titer (amount) and type (IgG, IgM, IgA) of antibody. This test allows for a better measure of the strength of the humoral response. In general, the higher the antibody titer the better the protection. However, the ELISA assays are more complex and can only be performed in a laboratory setting but not POC use.

Currently, a number of promising vaccines are under active development and in clinical trials. For most of them, the key is to train the immune system to generate neutralizing antibodies (nAbs) that recognize SARS-CoV-2's S protein and block its cellular entry via binding to the ACE2 cell receptor. Indeed, plasma derived from human convalescents and replete with nAbs has shown early promise as a COVID-19 treatment. The quality and quantity of the antibody response dictate functional outcomes. For example, in the case of SARS-CoV, viral docking on ACE2 on host cells is blocked when nAbs recognize the RBD domain or the heptad repeat 2 (HR2) domain on the S protein. In addition, nAbs can interact with other immune components, including phagocytes and natural killer cells, to assist pathogen clearance. However, sub-optimal pathogen-specific antibodies can promote pathology in some cases, resulting in a phenomenon known as antibody-dependent enhancement (ADE). Multiple factors determine whether an antibody neutralizes a virus or causes ADE and acute inflammation, including the specificity, concentration, affinity and isotype of the antibody. For example, in vitro data suggest that ADE occurs when antibody is present at a low concentration but dampens at the high-concentration range.

Although vaccines encoding SARS-CoV S protein and nucleocapsid (N) protein both provoke anti-S and anti-N IgG in immunized mice to a similar extent, N protein-immunized mice show significant upregulation of pro-inflammatory cytokine secretion and more severe lung pathology. Similarly, antibodies targeting different epitopes on S protein may vary in their potential to induce neutralization or ADE. For instance, antibodies reactive to the RBD domain or the HR2 domain of the S protein induce better protective antibody responses in non-human primates, whereas antibodies specific for other S protein epitopes can induce ADE. It is reported that the recombinant SARS-CoV-2 RBD antigen is highly sensitive and specific for detection of antibodies induced by SARS-CoVs. Further, a strong correlation was observed between the levels of RBD-binding antibodies and levels of SARS-CoV-2 neutralizing antibodies in patients. It was also found that only RBD-binding nAbs showed SARS-CoV-2 pseudovirus neutralization effects, and only nAbs bound to the RBD with a kD smaller or close to the dissociation constant of ACE2/RBD (15.9 nM) would have significant neutralization effects. These results support the use of RBD-based antibody assays for serology and as a correlate of neutralizing antibody levels in people who have recovered from infections or vaccinated. In addition to targeting the RBD of SARS-CoV-2, the compositions, assays, and systems described herein can be used with any SARS-CoV-2 antigen recognized by antibodies in a sample, as would be recognized by one of ordinary skill in the art based on the present disclosure.

Neutralizing assay is important toward evaluating the effectiveness of nAbs in blocking the viral infection. The gold standard is viral plaque reduction neutralization assay, where viruses replicate inside cells grown in cultures and are subsequently released when the cells are lysed or killed. This assay measures not only the titer of the antibody but also its ability to protect against viral infection. However, these assays are very labour intensive and must be performed in biosafety level 3 (BSL3) labs. Given limited access to BSL-3 facilities, researchers have turned to surrogate viral systems. These include retroviruses, lentiviruses, or replication-defective pseudoviruses with SARS-CoV-2 S protein and other molecular competent for a single round of viral entry and infection. However, these pseudotyped viruses are typically laborious to produce and challenging to scale up. Currently, there is still a lack of easy-to-use, inexpensive and accurate neutralization assays, which are important for drug discovery, vaccine development, and patient treatment with donated convalescent plasma.

Based on the above, embodiments of the present disclosure combine experimental analysis with simple physical interpretations and a theoretical model to comprehensively study the mechanisms of MNP-based multivalent analyte-binding in antibody sensing, and evaluate the assay performance in limit of detection, specificity, assay time, etc. In some embodiments, assays comprising heterogeneous MNPs are provided, including sandwich assay formats for immunoglobulin sensing and competitive assay formats for nAb sensing. Detection systems with portable electronic readout capability can be used with the assays disclosed herein, and these systems will incorporate different assay variants (e.g., MNP in liquid phase (in microcentrifuge tubes or customized polydimethylsiloxane (PDMS) well plate) and dried state (on glass or gold surface), for bare-eye readout, spectroscopic quantification, and optical and structural analysis).

Embodiments of the present disclosure include a liquid-phase sensing system to detect antibody-induced MNP concentration changes. Using gold nanoparticles (AuNPs), the AuNP monomers are initially uniformly dispersed, presenting a reddish color of the suspension in a microcentrifuge tube due to extinction from LSPR resonance (e.g., at around 560 nm for 80 nm particles). In one embodiment, the AuNPs can be surface-coated with streptavidin by first self-assembly thiolated carboxyl poly(ethylene glycol) linker via thiol-sulfide reaction and then functionalization of streptavidin via amine-carboxyl coupling by N-Hydroxysuccinimide/1-Ethyl-3-(3-dimethylaminopropyl) carbodiimide (NHS/EDC) chemistry. Then, biotinylated RBD will be mixed with AuNPs, followed by filtration to remove excessive RBD, to form the RBD rendered AuNPs for detection of virus-binding antibodies. This suspension of AuNPs is ready to detect immunoglobulins such as IgG via IgG-RBD binding, resulting in subsequent bridging of AuNP monomers to larger aggregates. These AuNP aggregates eventually precipitate driven by gravity. As a result, higher IgG concentrations would result in smaller concentration of AuNP monomers in the suspension and significant decrease in color intensity (or saturation). For example, the color contrast between 100 nM or 10 nM IgG samples and a reference (PBS buffer in lieu of IgG) can be easily recognized by naked eye or imaging (e.g., using a smartphone).

Naked eye readout is very useful for semi-quantitative diagnostics, but more accurate quantification requires more careful analysis of the optical spectra of the assay liquid. Embodiments of the present disclosure include the use of a customized PDMS well plate as a sample cuvette to obtain improved accuracy. The PDMS wells can be designed into different thicknesses (through curing in a petri-dish) and diameters (by punchers) and bonded to a glass slides after solvent cleaning and oxygen plasma treatment. This well plate can be sealed with a cover glass to avoid solution evaporation, and readily examined using a UV-visible spectrometer coupled to an upright microscope for spectral readout.

### 4. Detection Systems

Embodiments of the present disclosure also include sensing systems and corresponding kits incorporating the compositions and assays described herein. For example, in some embodiments, the systems and kits of the present disclosure include a device for measuring and/or detecting antibodies using the assays described herein. In some embodiments, systems will be capable of detecting and/or measuring SARS-CoV-2 antibodies with a time period of minutes.

Embodiments of the present disclosure also include a portable and integratable electronic readout system. In some embodiments, a portable detector that quantifies MNP suspension color will be used to determine a precise readout. Here a pair of low-cost LEDs and photodetectors will be attached to a 3D-printed microcentrifuge tube holder for miniaturized system integration. The basic working principle is simple: a LED emits narrow-band light at the MNP extinction wavelength, which is strongly absorbed and scattered by MNPs in the centrifuge tube, and the transmitted light will be then collected by a photodetector and read out as either the photodetector current or voltage on a serial resistor. The technology has a few advantages compared to the spectroscopic readout. First, the LEDs, photodetectors, as well as other electronic components (such as batteries, resistors and ammeters, or voltmeters) are large-scale manufacturable and commercially available at very low cost. (For example, green LEDs cost $0.50 each and photodetectors cost $1.40 each.) This can significantly lower the cost of the sensing system and make it much more easily accessible. Second, these electronic components have very small foot-print (typically a few millimeters to one centimeter) and can be easily integrated into a portable and light-weight readout device. This will greatly facilitate its use in point-of-care applications. For example, the sizes of LEDs and photodetectors are comparable to the diameter of an electric wire welded to them. Further integration of multiple such LED/photodetector pairs is feasible onto printed circuit board for compact and multiplexed readout. Third, the electronic readout is much more accurate than bare-eye readout, and is accessible to anyone, including those who face challenges in color perception. Fourth, the electronic readout can be readily stored into computers or online database, saving time for data management and making the data available for long period of time.

Additional details related to compositions and methods for rapid viral detection that are optionally adapted for use with the methods and related aspects disclosed herein are disclosed in U.S. patent application Ser. No. 17/532,969, filed Nov. 22, 2021 (Pub. No. US 2022/0163522 A1), which is incorporated by reference in its entirety.

Some further aspects are defined in the following clauses:

Clause 1: A method of analyzing a sample. The method comprising: contacting the sample with a first plurality of plasmonic metal nanoparticles (MNPs) having a viral antigen bound to its surface and a second plurality of MNPs having a viral antigen binding moiety bound to its surface to form a binding composition, wherein the viral antigen comprises at least one target epitope and wherein the viral antigen binding moiety specifically binds to the target epitope; and detecting a detectable signal from the binding composition that is indicative of the presence of a neutralizing antibody in the sample, the presence of a non-neutralizing antibody in the sample, or the absence of the neutralizing or the non-neutralizing antibody in the sample, thereby analyzing the sample.

Clause 2: The method of Clause 1, wherein the first and second pluralities of MNPs comprise a size and shape suitable for colorimetric, spectrometric, or electronic detection.

Clause 3: The method of Clause 1 or Clause 2, wherein the first and second plurality of MNPs differ from one another.

Clause 4: The method of any one of the preceding Clauses 1-3, wherein the viral antigen comprises a dimer or oligomer structure that comprises two or more copies of the viral antigen.

Clause 5: The method of any one of the preceding Clauses 1-4, further comprising determining a quantity or a concentration of the neutralizing antibody in the sample when the detectable signal from the binding composition is indicative of the presence of the neutralizing antibody in the sample.

Clause 6: The method of any one of the preceding Clauses 1-5, further comprising determining a quantity or a concentration of the non-neutralizing antibody in the sample when the detectable signal from the binding composition is indicative of the presence of the non-neutralizing antibody in the sample.

Clause 7: The method of any one of the preceding Clauses 1-6, further comprising obtaining a sample from a subject prior to or concurrent with the contacting step.

Clause 8: The method of any one of the preceding Clauses 1-7, wherein the sample is not processed prior to the contacting step.

Clause 9: The method of any one of the preceding Clauses 1-8, wherein the viral antigen is a SARS-CoV-2 antigen.

Clause 10: The method of any one of the preceding Clauses 1-9, wherein the SARS-CoV-2 antigen comprises an S1 subunit or receptor binding domain (RBD) of the spike (S) protein, or the S protein, or the N protein, or a fragment thereof.

Clause 11: The method of any one of the preceding Clauses 1-10, wherein the SARS-CoV-2 antigen comprises an artificially designed, dimer or oligomer form of an S1 subunit or RBD of the spike (S) protein, or the S protein, or the N protein, or a fragment thereof.

Clause 12: The method of any one of the preceding Clauses 1-11, wherein the viral antigen binding moiety is a SARS-CoV-2 antigen binding moiety.

Clause 13: The method of any one of the preceding Clauses 1-12, wherein the SARS-CoV-2 antigen binding moiety comprises an angiotensin-converting enzyme 2 (ACE2), or a fragment thereof.

Clause 14: A method of analyzing a sample. The method comprising: contacting the sample with a first plurality of plasmonic metal nanoparticles (MNPs) having a viral antigen bound to its surface and a second plurality of MNPs having a viral antigen binding moiety bound to its surface to form a binding composition, wherein the viral antigen comprises at least one target epitope and wherein the viral antigen binding moiety specifically binds to the target epitope; and detecting a first, a second, or a third MNP extinction wavelength from the binding composition, wherein the first MNP extinction wavelength is indicative of the presence of a neutralizing antibody in the sample, wherein the second MNP extinction wavelength is indicative of the presence of a non-neutralizing antibody in the sample and wherein the third MNP extinction wavelength is indicative of the absence of the neutralizing or the non-neutralizing antibody in the sample, thereby analyzing the sample.

Clause 15: The method of Clause 14, wherein the first and second plurality of MNPs differ from one another.

Clause 16: The method of Clause 14 or Clause 15, wherein the viral antigen comprises a dimer or oligomer structure that comprises two or more copies of the viral antigen.

Clause 17: The method of any one of the preceding Clauses 14-16, further comprising determining a quantity or a concentration of the neutralizing antibody in the sample when the first MNP extinction wavelength is detected from the binding composition or determining a quantity or a concentration of the non-neutralizing antibody in the sample when the second MNP extinction wavelength is detected from the binding composition.

Clause 18: The method of any one of the preceding Clauses 14-17, further comprising obtaining a sample from a subject prior to or concurrent with the contacting step.

Clause 19: The method of any one of the preceding Clauses 14-18, wherein the sample is not processed prior to the contacting step.

Clause 20: The method of any one of the preceding Clauses 14-19, wherein the viral antigen is a SARS-CoV-2 antigen.

Clause 21: The method of any one of the preceding Clauses 14-20, wherein the SARS-CoV-2 antigen comprises an S1 subunit or receptor binding domain (RBD) of the spike (S) protein, or the S protein, or the N protein, or a fragment thereof.

Clause 22: The method of any one of the preceding Clauses 14-21, wherein the SARS-CoV-2 antigen comprises an artificially designed, dimer or oligomer form of an S1 subunit or RBD of the spike (S) protein, or the S protein, or the N protein, or a fragment thereof.

Clause 23: The method of any one of the preceding Clauses 14-22, wherein the viral antigen binding moiety is a SARS-CoV-2 antigen binding moiety.

Clause 24: The method of any one of the preceding Clauses 14-23, wherein the SARS-CoV-2 antigen binding moiety comprises an angiotensin-converting enzyme 2 (ACE2), or a fragment thereof.

Clause 25: A method of analyzing an antibody in a sample. The method comprising: contacting a first aliquot of the sample with a first plurality of plasmonic metal nanoparticles (MNPs) having a first viral antigen variant bound to its surface and a second plurality of MNPs having a viral antigen binding moiety bound to its surface to form a first binding composition, wherein the viral antigen binding moiety binds to the first viral antigen variant; contacting a second aliquot of the sample with a third plurality of MNPs having a second viral antigen variant bound to its surface and the second plurality of MNPs having the viral antigen binding moiety bound to its surface to form a second binding composition, wherein the viral antigen binding moiety binds to the second viral antigen variant; and detecting a first MNP extinction wavelength from the first binding composition and a second MNP extinction wavelength from the second binding composition, wherein the first MNP extinction wavelength is indicative of a level of interaction of the antibody with the first viral antigen variant and wherein the second MNP extinction wavelength is indicative of a level of interaction of the antibody with the second viral antigen variant, thereby analyzing the antibody in the sample.

Clause 26: The method of Clause 25, wherein the first, second, and/or third plurality of MNPs differ from one another.

Clause 27: The method of Clause 25 or Clause 26, wherein the first and/or second viral antigen comprises a dimer or oligomer structure that comprises two or more copies of the first and/or second viral antigen.

Clause 28: The method of any one of the preceding Clauses 25-27, further comprising determining a quantity or a concentration of the neutralizing antibody in the sample when the first MNP extinction wavelength is detected from the binding composition or determining a quantity or a concentration of the non-neutralizing antibody in the sample when the second MNP extinction wavelength is detected from the binding composition.

Clause 29: The method of any one of the preceding Clauses 25-28, further comprising obtaining a sample from a subject prior to or concurrent with the contacting step.

Clause 30: The method of any one of the preceding Clauses 25-29, wherein the sample is not processed prior to the contacting step.

Clause 31: The method of any one of the preceding Clauses 25-30, wherein the first and/or second viral antigen is a SARS-CoV-2 antigen.

Clause 32: The method of any one of the preceding Clauses 25-31, wherein the SARS-CoV-2 antigen comprises an S1 subunit or receptor binding domain (RBD) of the spike (S) protein, or the S protein, or the N protein, or a fragment thereof.

Clause 33: The method of any one of the preceding Clauses 25-32, wherein the SARS-CoV-2 antigen comprises an artificially designed, dimer or oligomer form of an S1 subunit or RBD of the spike (S) protein, or the S protein, or the N protein, or a fragment thereof.

Clause 34: The method of any one of the preceding Clauses 25-33, wherein the viral antigen binding moiety is a SARS-CoV-2 antigen binding moiety.

Clause 35: The method of any one of the preceding Clauses 25-34, wherein the SARS-CoV-2 antigen binding moiety comprises an angiotensin-converting enzyme 2 (ACE2), or a fragment thereof.

Clause 36: A method of analyzing an antibody in a sample. The method comprising: contacting the sample with a plurality of plasmonic metal nanoparticles (MNPs) having a viral antigen binding moiety bound to its surface and viral antigen that comprises two or more epitopes that bind to the viral antigen binding moiety to form a binding composition; and detecting an MNP extinction wavelength from the binding composition, which MNP extinction wavelength is indicative of a level of interaction of the antibody with the viral antigen, thereby analyzing the antibody in the sample.

Clause 37: The method of Clause 36, wherein the two or more epitopes are identical to one another.

Clause 38: The method of Clause 36 and Clause 37, wherein the two or more epitopes are different from one another.

Clause 39: The method of any one of the preceding Clauses 36-38, wherein the viral antigen comprises a dimer or oligomer structure that comprises two or more copies of the viral antigen.

Clause 40: The method of any one of the preceding Clauses 36-39, further comprising determining a quantity or a concentration of the antibody in the sample.

Clause 41: The method of any one of the preceding Clauses 36-40, further comprising obtaining a sample from a subject prior to or concurrent with the contacting step.

Clause 42: The method of any one of the preceding Clauses 36-41, wherein the sample is not processed prior to the contacting step.

Clause 43: The method of any one of the preceding Clauses 36-42, wherein the viral antigen is a SARS-CoV-2 antigen.

Clause 44: The method of any one of the preceding Clauses 36-43, wherein the SARS-CoV-2 antigen comprises an S1 subunit or receptor binding domain (RBD) of the spike (S) protein, or the S protein, or the N protein, or a fragment thereof.

Clause 45: The method of any one of the preceding Clauses 36-44, wherein the SARS-CoV-2 antigen comprises an artificially designed, dimer or oligomer form of an S1 subunit or RBD of the spike (S) protein, or the S protein, or the N protein, or a fragment thereof.

Clause 46: The method of any one of the preceding Clauses 36-45, wherein the viral antigen binding moiety is a SARS-CoV-2 antigen binding moiety.

Clause 47: The method of any one of the preceding Clauses 36-46, wherein the SARS-CoV-2 antigen binding moiety comprises an angiotensin-converting enzyme 2 (ACE2), or a fragment thereof.

Clause 48: A system, comprising: a receptacle receiving area configured to receive at least one receptacle that comprises the binding composition of any one of the preceding Clauses 1-13; and a detector capable of detecting the detectable signal from the binding composition of any one of the preceding Clauses 1-13.

Clause 49: A system, comprising: a receptacle receiving area configured to receive at least one receptacle that comprises the binding composition of any one of the preceding Clauses 14-24; a light source capable of emitting photons having an MNP extinction wavelength corresponding to one or more of the pluralities of MNPs of any one of the preceding Clauses 14-24; and a photodetector capable of detecting transmitted light from one or more of the pluralities of MNPs of any one of the preceding Clauses 14-24.

Clause 50: A system, comprising: a receptacle receiving area configured to receive at least one receptacle that comprises the first or second binding composition of any one of the preceding Clauses 25-35; a light source capable of emitting photons having an MNP extinction wavelength corresponding to one or more of the pluralities of MNPs of any one of the preceding Clauses 25-35; and a photodetector capable of detecting transmitted light from one or more of the pluralities of MNPs of any one of the preceding Clauses 25-35.

Clause 51: A system, comprising: a receptacle receiving area configured to receive at least one receptacle that comprises the binding composition of any one of the preceding Clauses 36-47; a light source capable of emitting photons having an MNP extinction wavelength corresponding to the plurality of MNPs of any one of the preceding Clauses 36-47; and a photodetector capable of detecting transmitted light from the plurality of MNPs of any one of the preceding Clauses 36-47.

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

What is claimed is:

1. A method of analyzing an antibody in a sample, the method comprising:
contacting the sample with a plurality of plasmonic metal nanoparticles (MNPs) having a viral antigen binding moiety bound to its surface and viral antigen that comprises two or more epitopes that bind to the viral antigen binding moiety to form a binding composition; and,
detecting an MNP extinction wavelength from the binding composition, which MNP extinction wavelength is indicative of a level of interaction of the antibody with the viral antigen, thereby analyzing the antibody in the sample.

2. The method of claim 1, wherein the two or more epitopes are identical to one another.

3. The method of claim 1, wherein the two or more epitopes are different from one another.

4. The method of claim 1, wherein the viral antigen comprises a dimer or oligomer structure that comprises two or more copies of the viral antigen.

5. The method of claim 1, further comprising determining a quantity or a concentration of the antibody in the sample.

6. The method of claim 1, further comprising obtaining a sample from a subject prior to or concurrent with the contacting step.

7. The method of claim 1, wherein the sample is not processed prior to the contacting step.

8. The method of claim 1, wherein the viral antigen is a SARS-CoV-2 antigen.

9. The method of claim 8, wherein the SARS-CoV-2 antigen comprises an S1 subunit or receptor binding domain (RBD) of the spike (S) protein, or the S protein, or the N protein, or a fragment thereof.

10. The method of claim 8, wherein the SARS-CoV-2 antigen comprises an artificially designed, dimer or oligomer form of an S1 subunit or RBD of the spike (S) protein, or the S protein, or the N protein, or a fragment thereof.

11. The method of claim 1, wherein the viral antigen binding moiety is a SARS-CoV-2 antigen binding moiety.

12. The method of claim 11, wherein the SARS-CoV-2 antigen binding moiety comprises an angiotensin-converting enzyme 2 (ACE2), or a fragment thereof.

13. A method of analyzing a sample, the method comprising:
contacting the sample with a first plurality of plasmonic metal nanoparticles (MNPs) having a viral antigen bound to its surface and a second plurality of MNPs having a viral antigen binding moiety bound to its surface to form a binding composition, wherein the viral antigen comprises at least one target epitope and wherein the viral antigen binding moiety specifically binds to the target epitope; and,
detecting a first, a second, or a third MNP extinction wavelength from the binding composition, wherein the first MNP extinction wavelength is indicative of the presence of a neutralizing antibody in the sample, wherein the second MNP extinction wavelength is indicative of the presence of a non-neutralizing antibody in the sample and wherein the third MNP extinction wavelength is indicative of the absence of the neutralizing or the non-neutralizing antibody in the sample, thereby analyzing the sample.

14. The method of claim 13, wherein the first and second plurality of MNPs differ from one another.

15. The method of claim 13, wherein the viral antigen comprises a dimer or oligomer structure that comprises two or more copies of the viral antigen.

16. The method of claim 13, further comprising determining a quantity or a concentration of the neutralizing antibody in the sample when the first MNP extinction wavelength is detected from the binding composition or determining a quantity or a concentration of the non-neutralizing antibody in the sample when the second MNP extinction wavelength is detected from the binding composition.

17. The method of claim 13, further comprising obtaining a sample from a subject prior to or concurrent with the contacting step.

18. The method of claim 13, wherein the viral antigen is a SARS-CoV-2 antigen.

19. The method of claim 13, wherein the viral antigen binding moiety is a SARS-CoV-2 antigen binding moiety.

20. A system, comprising:
a receptacle receiving area configured to receive at least one receptacle that comprises a binding composition comprising a plurality of plasmonic metal nanoparticles (MNPs) having a viral antigen binding moiety bound to its surface and viral antigen that comprises two or more epitopes that bind to the viral antigen binding moiety;
a light source capable of emitting photons having an MNP extinction wavelength corresponding to the plurality of MNPs; and
a detector capable of detecting transmitted light from the plurality of MNPs.
